(19) United States
(12) Patent Application Publication
(10) Pub. No.: US 2026/0131108 A1
(43) Pub. Date: May 14, 2026

(54) PACKAGED CATHETER ASSEMBLY

(71) Applicant: CONVATEC LIMITED, Flintshire (GB)

(72) Inventors: Gavin ROBERTS, Swindon (GB); Nicola PILMER, Chester (GB); Alice YOUNG, Chester (GB)

(21) Appl. No.: 19/390,619

(22) Filed: Nov. 16, 2025

Related U.S. Application Data
(63) Continuation of application No. PCT/GB2024/051270, filed on May 15, 2024.
(60) Provisional application No. 63/502,902, filed on May 17, 2023.

(30) Foreign Application Priority Data
Jul. 18, 2023 (GB) 2310986.1

(51) Int. Cl. A61M 25/00

(52) U.S. Cl. CPC A61M 25/002; A61M 2202/0014; A61M 2202/04; A61M 2207/00; A61M 2209/06

(57) ABSTRACT

The present disclosure relates to a packaged catheter assembly that has a catheter (102) and a fluid collection bag (101) in packaging (1). The fluid collection bag (101) is releasably retained in the packaging (1) by a strap (50). Further, the strap (50) may be joined to the packaging (1).

### TECHNICAL FIELD OF THE INVENTION

The present invention relates to packaged catheter assemblies. In particular the invention concerns packaged urinary catheter assemblies, and most particularly, but not exclusively packaged intermittent male urinary catheter assemblies.

### BACKGROUND TO THE INVENTION

Many people suffer from urinary problems that can make it difficult to pass urine. Intermittent catheter assemblies can provide a convenient and portable solution to this problem as a user is able to self-catheterise to relieve themselves as required. This reduces the effect of their urinary condition on their life and allows them to enjoy a relatively normal lifestyle.

It is desirable that catheter assemblies are discreet such that a layperson may not identify them as a packaged catheter assembly at all. This can make a user feel more comfortable carrying and using them. However, along with being compact, the packaged catheter assembly must remain usable such as by providing visual feedback of the drain of fluid from the body. This can make providing a discreet packaged catheter assembly that is also easy to use difficult and so a user may decide to place the packaged catheter assembly in a dedicated bag to hide it. However, this can cause hygiene issues if the bag is not cleaned/replaced regularly and also increase the weight and bulk of the packaged catheter assembly when carried around.

In order to make the packaging as discrete as possible it is desirable to make the packaging as small, conforming to the shape of the catheter as much as possible. This can make insertion of the catheter assembly into the packaging during assembly difficult.

It is an object of embodiments of the present invention to at least partially overcome or alleviate the above problems and/or to provide an improved packaged catheter assembly.

### SUMMARY OF THE INVENTION

In broad terms, the present invention concerns a packaged catheter assembly comprising the catheter packaging and a catheter assembly, wherein the packaging is configured to contain the catheter assembly within it.

According to one broad aspect of the present disclosure there is provided a packaged catheter assembly. The catheter assembly may comprise a catheter. The catheter assembly may comprise a fluid collection bag. The catheter tube and/or fluid collection bag may be in packaging. The fluid collection bag may be releasably retained in the packaging. The fluid collection bag may be releasably retained in the packaging by a handling/retention device. The handling/retention device may be joined to the packaging.

The handling/retention device may be a strap. The strap may be joined to the packaging.

As such, according to a first aspect of the present disclosure there is provided a packaged catheter assembly comprising a catheter and a fluid collection bag in packaging; wherein the fluid collection bag is releasably retained in the packaging by a strap; the strap being joined to the packaging.

By providing a strap to releasably retain the fluid collection bag, packaging the catheter assembly during manufacture is made easier as the position of the fluid collection bag can be controlled. Furthermore, by joining the strap to the packaging, removal of the catheter assembly does not result in removal of the strap which avoids the strap having to be disposed of separately or become lost, and allows the catheter to be ready to use on removal.

The strap may releasably retain the catheter. By also retaining the catheter, the strap can ensure that the orientation of the catheter does not change during storage and transport. This mean a handling surface for removing the catheter assembly from the packaging can be presented proximate to an opening of the packaging thereby reducing the risk of contamination of the catheter on removal. The strap may retain the fluid collection bag in a folded (or rolled) configuration. By retaining the fluid collection bag in a folded (or rolled) configuration the amount of space taken up by the fluid collection bag is reduced thereby reducing the size of the packaging, the strap ensuing the folded configuration is maintained. The strap may comprise a plastics material. For example, polypropylene (PP), polyethylene terephthalate (PET), low density polyethylene (LDPE), metalized polyester (MET PET), polyvinyl chloride (PVC) or orientated polypropylene (OPP). The strap may comprise polyethylene (PE).

The packaging may comprise a pouch. The pouch may comprise a flexible plastics material, such as a flexible plastic sheet or film. For example, polypropylene (PP), polyethylene terephthalate (PET), low density polyethylene (LDPE), metalized polyester (MET PET), polyvinyl chloride (PVC) or orientated polypropylene (OPP).

The pouch may be defined by two walls joined at their periphery by a peripheral join. The peripheral join may comprise any one or more of: a weld; mechanical seal; heat seal; pressure seal; adhesive; solvent bond; ultraviolet bond; ultrasonic weld; laser weld; impulse weld; or friction weld. The peripheral join may provide a sterile seal.

The strap may be bonded to an internal surface of a pouch wall. The strap may be welded to an internal surface of a pouch wall. The strap may be joined to the peripheral join. The strap may be configured to be joined in the same step as the peripheral join. As such, where the peripheral join comprises a weld, the strap may be welded with the peripheral join. Having the strap join at the peripheral join makes manufacture simpler as a single step can be used to form the pouch and secure the strap.

The strap may be an elongate rectangular shape. The strap may extend along a longitudinal axis. The strap may comprise a first end and a second end. The first end may be bonded (e.g. welded) to the internal surface of the pouch wall. The first end may be bonded (e.g. welded) to the peripheral join. The second end may comprise a first engaging means. A second, corresponding, engaging means may be provided between the two ends. The two engaging means may be configured to engage to form the stap into a loop. The engaging means may comprise corresponding slots and tabs. The engaging means may comprise corresponding hooks and fasteners. One of the first and second engaging means may comprise a rectangular slot perpendicular to the longitudinal axis. The other of the first and second engaging means may comprise a U-shaped slot defining a tab.

The peripheral join may be provided on two lateral edges of the pouch. The pouch may further comprise an openable top edge. The pouch may further comprise a sealed base edge.

The two walls may comprise a flexible plastics material. For example, polypropylene (PP), polyethylene terephthalate (PET), low density polyethylene (LDPE), metalized polyester (MET PET) or orientated polypropylene (OPP). The two walls may be formed from a single sheet of material.

The two walls may be unbonded beyond the top seal. The two walls may be unbonded above the top seal. The two walls may extend beyond the top seal. A portion of each wall above the top seal may form a flap. A portion of each wall beyond the top seal may form a flap. There may be two flaps. A distal end of the flap may define an upper edge of the packaging. One or both flaps may be provided with a finger hole. The finger hole may extend through the respective wall. Both finger holes may be sized to allow a user's finger to be at least partially inserted through. Both finger holes may be a circle, an oval, an ellipse, a polygon, a square, a rounded square, a stadium, a triangle, a rounded triangle.

The lateral edge bonds may comprise a frangible portion. The frangible portion of the lateral edge bonds may be proximate to the top seal. The lateral edge bond may comprise a tear stop. The tear stop may delimit the frangible portion of the lateral edge bonds from a non-frangible portion of the lateral edge bonds. The tear stop may comprise a widened portion of the lateral edge bond. The tear stop may be at least twice as wide as the lateral edge bond. The tear stop may be bonded with a stronger bonding technique than the frangible portion of the lateral edge bond. The tear stop may require a separation force at least twice the separation for the frangible portion of the lateral edge bond. The tear stop may be arranged about 80 mm above the base edge. The top seal may be frangible.

The strap may be joined to one of the lateral edge bonds between the tear stop and the base edge. The strap may be configured to be retained in the packaging when the catheter assembly is removed. Joining the strap to the packaging between the tear stop and the base edge of the packaging ensures the strap remains secure when the packaging is opened.

The strap may be arranged closer to the tear stop than the base edge. The strap may be arranged no more than 50%, 40%, 30%, 25% or 20% of the way along the lateral edge bond from the tear stop to the base edge. Preferably, the strap is arranged about 25% of the way along the lateral edge bond from the tear stop to the base edge. The strap may be arranged no more than 10 mm, 20 mm or 30 mm from the tear stop. The strap is preferably arranged about 20 mm from the tear stop. The strap may be arranged at least 20 mm, 40 mm or 60 mm from the base edge. Preferably, the strap is arranged about 60 mm from the base edge. This ensures the catheter assembly is held in a more convenient location for removal from the packaging.

The packaging may be a sterile packaging. That is when sealed the packaging provides a sterile cavity within.

According to a further broad aspect of the disclosure there is provided a method of packaging a catheter assembly. The catheter assembly may comprise a catheter tube. The catheter assembly may comprise a fluid collecting bag. The method may comprise securing the fluid collection bag with a strap. The method may comprise joining the strap to an interior of packaging.

Accordingly in a second aspect of the disclosure there is provided a method of packaging a catheter assembly comprising a catheter tube and a fluid collection bag, the method comprising; securing the fluid collection bag with a strap and joining the strap to an interior wall of packaging.

The packaged catheter assembly of the second aspect may be the packaged catheter assembly of the first aspect and may include any optional features thereof.

The method may further comprise sealing the packaging around the catheter assembly. The packaging may be sealed by welding. The step of sealing the packaging around the catheter assembly may also join the strap to the packaging. The strap may be welded to the packaging.

The fluid collection bag may be folded prior to being secured.

The method may also comprise securing the catheter tube with the strap.

### DETAILED DESCRIPTION OF THE INVENTION

In order that the invention may be more clearly understood one or more embodiments thereof will now be described, by way of example only, with reference to the accompanying drawings, of which:

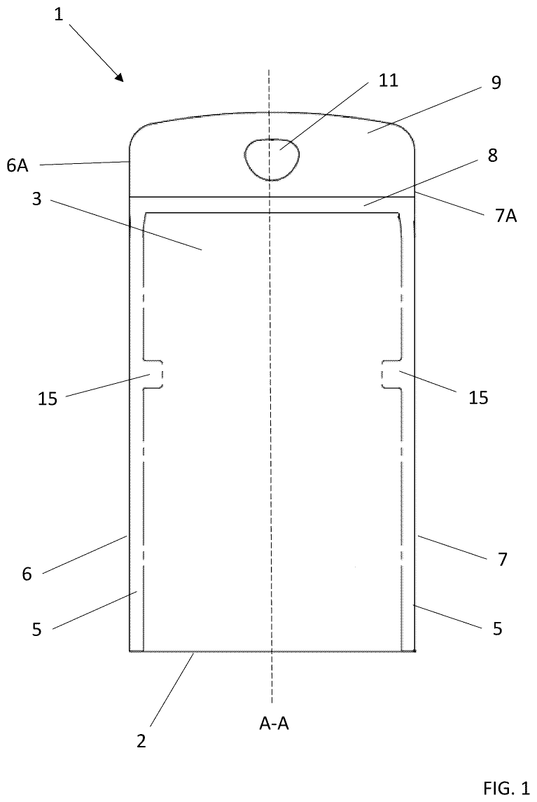

FIG. 1 is a front view of a first embodiment of a catheter packaging;

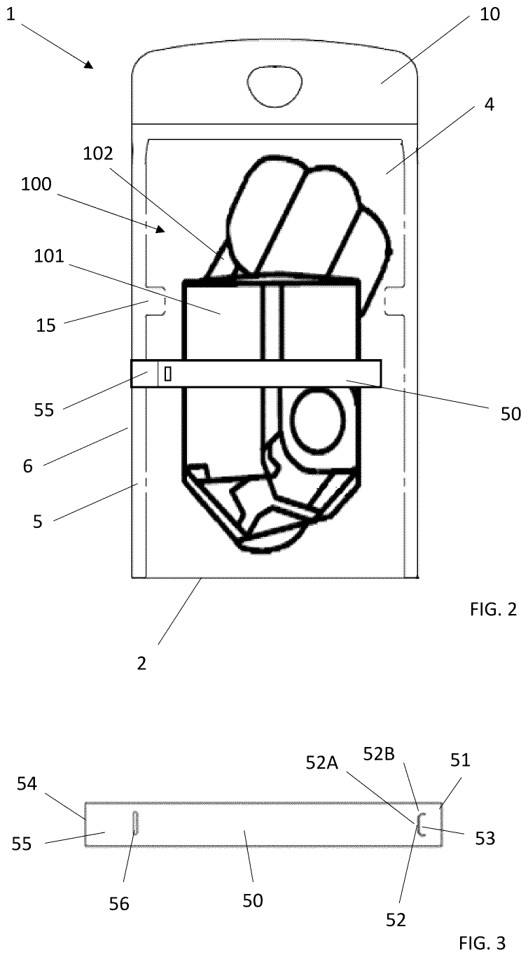

FIG. 2 is a front view of the catheter packaging of FIG. 1 with the front wall removed;

FIG. 3 is a front view of the retention strap of the catheter packaging of FIG. 1; and

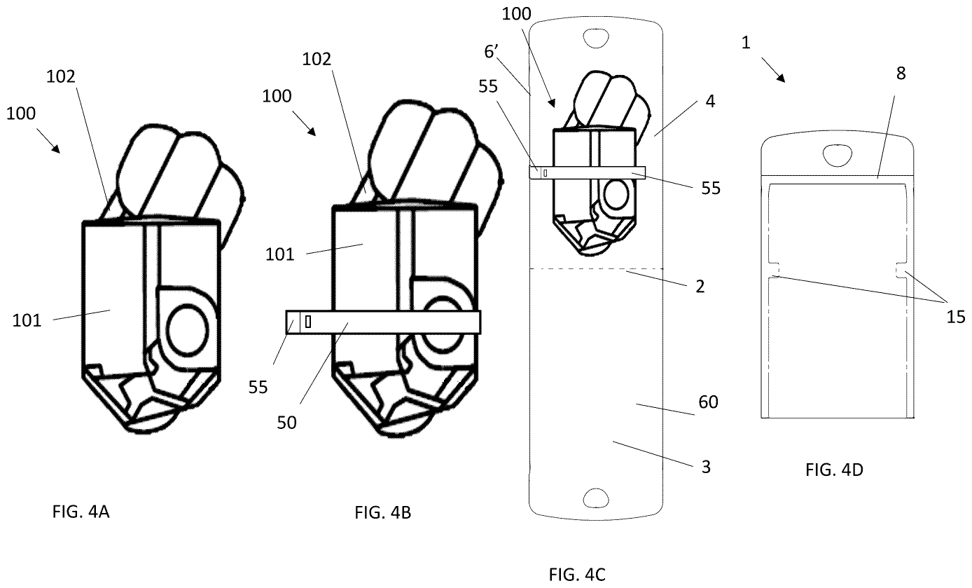

FIGS. 4a-d show a perspective view of the steps of packaging a catheter assembly according to an aspect of the invention.

In the figures, as is conventional, broken lines show hidden features.

Referring to FIGS. 1 and 2, an embodiment of a catheter packaging 1 is shown. In this embodiment the packaging 1 is formed from a single piece of material. The packaging 1 is formed by folding this single piece of material along a fold-line 2 spanning the width of the packaging at a point midway along its length to form a front wall 3 and rear wall 4 of the packaging. Lateral edge bonds 5 bond the front 3 and rear walls 4 along their lateral edges and a top seal 8 is provided between the two lateral edge bonds opposite the fold line 2. To aid in the description of the packaging, a longitudinal axis can be considered to extend along a centreline A-A of the packaging from the fold line 2 to the top seal 8.

In this embodiment, the packaging fold-line 2 forms a base edge 2 of the packaging. In this embodiment, the lateral edge bonds 5 form a left lateral edge and a right lateral edge. The left lateral edge 6 and right lateral edge 7 being defined as the right and left sides of the packaging 1 when viewing the packaging 1 with the rear wall 4 behind the front wall 3, the base edge 2 at the bottom of the packaging 1 and the top seal 8 at the top of the packaging 1.

In this embodiment, lateral edge bonds 5 and top seal 8 are formed through heat sealing the front wall 3 to the second wall 4 with a bond width of 5 mm, but in other embodiments any suitable bonding may be used such as Velcro (RTM), chemical adhesives, chemical bonding, ultrasonic bonding, or mechanical bonding. It is also conceivable that no folds may be provided, for example the front 3 and rear 4 walls may be provided separate sheets of material, in place of a fold line the two walls 3,4 may also be joined along the base edge 2 with heat sealing or other suitable bonding.

In this embodiment, the top seal 8 and a portion of the lateral edge bonds are configured to break to allow the packaging 1 to be opened. Consequently, the packaging 1 has a closed configuration in which it is sealed, and an open configuration in which an openable top is open and a catheter assembly within is accessible.

In this embodiment, the packaging 1 is formed from an opaque material and as such provides a discreet package for the packaged catheter assembly which can make the user more comfortable in carrying the packaged catheter assembly in day-to-day life. In addition, the exterior of the packaging 1 may have a textured or printed appearance and feel, such as a matte finish to reduce the impression that it is a medical device.

In this embodiment, a portion of the lateral edges 6,7 of the front and rear walls 3,4 are unbonded beyond the top seal 8. The unbonded portion of the front wall 3 forms a front opening tab 9 and the unbonded portion of the rear wall 4 forms a rear opening tab 10. Both the front opening tab 9 and rear opening tab 10 project from the packaging 1 along an edge corresponding to the top seal 8. Each of the tabs 9, 10 of this particular embodiment are rectangular in shape with lateral edges 6A, 7A parallel to and continuing from a respective lateral edge 6,7 of the packaging. At a midpoint of each tab 9,10 there is provided a finger hole 11. Both finger holes 11 have a rounded triangular shape which tapers towards the top seal 8. The top seal 8 may be provided by a bond line 8 extending perpendicular from the lateral edges 6,7.

The lateral edge bonds 5 may each be provided with a tear stop 15, the tear stops are arranged approximately two-thirds of the way along the lateral edge bonds 5 from the base 2 and comprise a reinforced portion of the lateral edge bond 5. In this embodiment the reinforcement is provided by increasing the width of the lateral edge bond 5, alternatively the tear stops 15 could be reinforced in other ways, for example a high temperature weld could be used or an adhesive applied. The position of the tear stops 15 determine the extent to which the cavity can be opened and the degree of access to the catheter assembly inside. In some embodiments the tear stops 15 are provided closer to the base 2, this improves the access the contents of the packaging, but also increases the risk the contents may accidentally spill. Conversely, the tear stops 15 could be provided further from the base, this would reduce the risk of accidentally dropping the contents, but also hinder their removal.

The base 2, lateral edge bonds 5 and top seal 8 together define a sterile cavity within the packaging in which a catheter assembly can be stored prior to use.

With reference to FIGS. 2 and 3 the interior of the packaging 1 is described. FIG. 2 shows the catheter packaging 1 with the front wall 3 removed to show the interior of the packaging. Within the sterile cavity there is provided a catheter assembly 100 comprising a fluid collection bag 101 and a catheter 102, in this embodiment the catheter is integrally formed with the fluid collection bag, and as such will be recognised as a closed catheter system, however it is envisioned that the invention could also be applied to open catheter systems. In order to reduce the size of the catheter assembly 100 when in a packaged configuration, the fluid collection bag 101 is provided in a folded configuration.

In order to retain the fluid collection bag 101 in the folder configuration a strap 50 (i.e. a retention/retaining strap 50) is provided around the fluid collection bag 101. In this embodiment the retention strap 50 is also provided around the catheter 102. As can be seen in FIG. 3, the retention strap 50 is an elongate rectangular strip. In the present embodiment the strip is formed from a sheet of polyethylene, although in alternative embodiments other flexible sheet materials could be used, such other plastics, paper or foils.

At a first end 51 of the strap 50 there is provided a U-shaped slit 52, the slit 52 comprises a long straight portion 52A parallel to the edge 51 and two shorter portions 52B which extend perpendicularly from the ends of the straight portion 52A towards the first end 51. The slit 52 thereby defines a tab 53 forming the first part 53 of an engaging means. At an opposing second end 54 of the strap 50 there is provided a bonding region 55, the purpose of which will be expanded upon below. Proximate to the bonding region 55 there is provided a second rectangular slit 56 extending parallel to the second end 54. The second slit 56 forms a second part 56 of the engaging means. The separation between the two slits 52,56 is approximately equal to, the distance around the catheter assembly 100 in the folded configuration. As will be appreciated by those skilled in the art the engaging means may be provided in alternative ways in order to allow the strap 50 to be formed into a loop. For example, the shapes of the slots can be varied or replaced by a contact adhesive or a hook and fastener arrangement.

The bonding region 55 of the retaining strap 50 is welded with the lateral edge bond 5 between the tear stop 15 and the base 2. This ensures that, as will be explained in greater detail below, when the packaging is opened and the catheter assembly is removed the retention strap is retained within the packaging thereby avoiding a separate component needing disposal. In alterative embodiments the retention strap 50 may be secured to the packaging in different locations and by different means, for example the bonding region may be bonded to any interior surface of the cavity by means such as adhesive. In one particular alternative embodiment the strap may be arranged on the lateral edge between the tear stop and the top-edge in doing so the strap may be provided with additional adhesive or similar joining means so as to ensure it retains attached when the packaging is opened.

In this embodiment, the strap 50 is arranged closer to the tear stop 15 than the base 2, such as about 25% of the way along the lateral edge bond 5 from the tear stop 15 to the base 2. This ensures the catheter assembly is held in a more convenient location for removal from the packaging. For example, the strap 50 may be attached about 20 mm below the tear stop 15 and/or about 60 mm above the base 2. The tear stop 15 may therefore be arranged about 80 mm above the base 2.

With reference to FIGS. 4a-d the method of assembling the packaged catheter according to an aspect of the invention is described.

As shown in FIG. 4a the fluid collection bag 101 is folded and the catheter 102 is brought against the flat surface of the fluid collection bag to bring the catheter assembly 100 into a folded configuration. Where the catheter assembly 100 is a closed catheter and the catheter is of sufficient length, any tubing connecting the catheter 102 to the fluid collection bag 101 may also be coiled within a footprint of the folded fluid collection bag 101, that is the folded fluid collection bag 101 defines the extent of the area taken by the coiled tubing, a handling surface 103 of the catheter may extend beyond the footprint of the folded fluid collection bag 101. Next, as shown in FIG. 4b, whilst the catheter assembly 100 is in the folded configuration the retention strap 50 is wrapped around it and the first 53 and second parts 56 of the engaging means are connected thereby securing the catheter assembly in the folded configuration.

The catheter assembly 100 secured by the retaining strap 50 is then placed on a sheet of material 60 which will form the packaging. The bonding region 55 is positioned such that it is aligned against a lateral edge 6′of the sheet approximately ⅓ of the way from the fold line 2 to a distal edge which will form the top edge 8. The catheter assembly 101 is positioned such that the handling surface 103 is proximate to the top edge 8. The sheet of material 60 is then folded over about the fold line 2 and a heated die is pressed against the folded sheet to weld the peripheral edge bonds 5, including the tear stops 15, and top seal 8. The welding of the peripheral edge bonds 5 also welds the bond region 55 of the retention strap 50 into the edge bond 5 thereby securing the retention strap 50 within the packaging 1 and thus the catheter assembly 100 within the packaging 1.

To use the catheter assembly the user or Health Care Professional (HCP) pulls the two opening tabs 9,10 apart which breaks the top seal 8 and continues to separate front and rear walls as the edge bond 5 breaks until the edge bond is broken to the tear stop 15. An opening is thus provided at the top of the packaging 1 allowing access to the catheter assembly. The user or HCP can then grip the catheter assembly 101 by the handling surface 103 and withdraw the catheter assembly 101 for use. Upon withdrawing the catheter assembly the engaging means 53,56 on the retaining strap breaks allowing the release of the catheter assembly, but due to the bonded region 55 being welded into the lateral edge bond 5 the retention strap is retained with the packaging and can be disposed of together.

The one or more embodiments are described above by way of example only. Many variations are possible without departing from the scope of protection afforded by the appended claims.

1. A packaged catheter assembly comprising a catheter and a fluid collection bag in packaging; wherein the fluid collection bag is releasably retained in the packaging by a strap, the strap being joined to the packaging.

2. The packaged catheter assembly according to claim 1 wherein the strap releasably retains the catheter.

3. The packaged catheter assembly according to claim 1 wherein the strap comprises a plastics material.

4. The packaged catheter assembly according claim 1, wherein the strap comprises polyethylene.

5. The packaged catheter assembly according to any preceding claim 1, wherein the strap is joined to an internal wall of the packaging.

6. The packaged catheter assembly according to claim 5 wherein the strap is joined to the packaging at a first end and a second end comprises a first engaging means, and a second corresponding engaging means is provided between the two ends, the engaging means configured to form the strap into a loop.

7. The packaged catheter assembly according to claim 1, wherein the packaging comprises a pouch defined by two walls joined at their periphery by a peripheral join, and wherein the strap is joined to the peripheral join.

8. The packaged catheter assembly according to claim 7, wherein the peripheral join is a weld and the strap is welded with the peripheral weld.

9. The packaged catheter assembly according to claim 7 wherein the peripheral join comprises a tear stop.

10. The packaged catheter assembly according to claim 9 wherein the pouch comprises two lateral edges, an openable top edge, and a sealed base edge, and wherein the strap is joined to the packaging on at least one of the lateral edges between the tear stop and the base edge.

11. The packaged catheter assembly according claim 1 wherein the packaging is a sterile packaging.

12. A method of packaging a catheter assembly comprising a catheter tube and a fluid collection bag, the method comprising:
securing the fluid collection bag with a strap; and
joining the strap to an interior wall of the packaging.

13. The method of packaging a catheter assembly according to claim 12, further comprising the step of sealing the packaging around the catheter assembly.

14. The method of packaging a catheter assembly according to claim 13, wherein sealing the packaging around the catheter assembly also joins the strap to the packaging.

15. The method of packaging a catheter assembly according to claim 13, wherein the packaging is sealed by welding.

16. The method of packaging a catheter assembly according to claim 13, wherein the packaging is sealed along a lateral edge and the strap is joined to the packaging at the lateral edge.

17. The method of packaging a catheter assembly according to claim 12, wherein the fluid collection bag is folded prior to being secured.

18. The method of packaging a catheter assembly according to claim 12, further comprising securing the catheter tube with the strap.

19. The method of packaging a catheter assembly according to claim 12, wherein the catheter packaging comprises the catheter tune and the fluid collection bag in packaging, wherein the fluid collection bag is releasably retained in the packaging by a strap, the strap being joined to the packaging.
